(19) United States
(12) Patent Application Publication
(10) Pub. No.: US 2019/0150838 A1
(43) Pub. Date: May 23, 2019

(54) METHOD AND APPARATUS FOR MEASUREMENT AND VISUAL FEEDBACK OF PHYSIOLOGIC SIGNALS THROUGH HEADWORN DEVICE

(71) Applicant: Devon Greco, Bend, OR (US)

(72) Inventor: Devon Greco, Bend, OR (US)

(21) Appl. No.: 16/037,297

(22) Filed: Jul. 17, 2018

Related U.S. Application Data
(60) Provisional application No. 62/533,413, filed on Jul. 17, 2017.

(51) Int. Cl. A61B 5/00; A61B 5/0476

(52) U.S. Cl. CPC A61B 5/6835; A61B 5/0476; A61B 5/6803

(57) ABSTRACT

An apparatus for obtaining EEG signals utilizes adjustable arms and EEG sensors mounted on an adjustable frame adapted to fit the head of an individual.

This application claims the benefit of U.S. Provisional Patent Appl. Ser. No. 62/533,413, filed Jul. 17, 2017, and incorporated herein by reference.

### BACKGROUND OF THE INVENTION

### Field of the Invention

The invention generally relates to the field of bio-signaling and more specifically to the field of biofeedback and neurofeedback for the encouragement of self-regulation of physiologic signals. This invention is related to the invention described in U.S. Patent U.S. Pat. No. 9,521,976 B2, priority date of Jan. 24, 2013.

### Description of Related Art

Many devices and techniques exist for the measurement of physiologic signals, and more specifically, of electroencephalography (EEG) for the purposes of research, diagnostics, and teaching self-regulation of said physiologic signals.

There is difficulty in offering a wearable sensing design that is comfortable, easy-to-use, and adjustable to different head sizes, as well as offering a wide range of 10-20 site placement compatibility in combination with active dry electrodes and passive visual feedback.

Some designs found in the prior art offer integrated dry electrodes and other designs offer integrated dry electrodes in combination with aural feedback, such as headphones, and further, other designs offer integrated dry electrodes in combination with visual feedback such as light emitting LEDs of various colors and intensity as a means of providing feedback.

None of the designs found in the prior art offer integrated dry electrodes that can be placed in various 10-20 site placements, in combination with electrochromic lens feedback which offer a comfortable and passive visual feedback which can be easily experienced while performing everyday activities.

One goal of such design is to incorporate both physiologic measurement and passive visual feedback in the form of auto-darkening electrochromic lenses, into the head-worn apparatus as well as offering adjustability to different head sizes as well as different 10-20 site placements.

### SUMMARY OF THE INVENTION

The invention generally relates to the field of bio-signaling and more specifically to the field of biofeedback and neurofeedback for the encouragement of self-regulation of physiologic signals.

The present invention includes a head-worn apparatus that incorporates physiologic measurement devices, such as EEG, as well as visual feedback in the form of variably darkening electrochromic lenses.

It is accordingly an objective of the invention to provide a biofeedback system and method that addresses the shortcomings of the prior art by incorporating both physiologic measurement and visual feedback into the head-worn apparatus as well as offering adjustability to different head sizes and various 10-20 site placements.

To accomplish this objective, the present invention provides a method and system for obtaining EEG signals through hair by way of an adjustable arm and connected dry active electrode with amplifier circuitry. Said adjustable arm and electrode with amplifier circuitry is mounted on an adjustable frame for fitment on the head. Said adjustable frame also contains electro-chromic lenses as described in the related U.S. Patent U.S. Pat. No. 9521976 B2.

The present invention also provides a method and system of transforming physiologic information obtained from biomedical instruments in order to use that information to modulate visual stimulation received by the user from the user's environment during normal interaction with that environment. The phrase “normal interaction with the environment” refers to interaction that would occur, or be carried out by the user, even in the absence of the biofeedback system and method.

In accordance with at least one embodiment of the present invention, the disclosed apparatus and methods can be used for safety, health, or productivity purposes. In one embodiment of the invention, physiologic signals related to stress, workload, or mental engagement could be used to control the lens opacity worn by a worker connected to the system. In one scenario, an individual would wear the device while reading. The physiologic data related to mental engagement would modulate the opacity of the electrochromic lens.

In one preferred embodiment of the present invention, dry active electrodes are used which enables the measurement of EEG without moisture or preparing the skin by cleaning and abrading.

In one aspect of the present invention, the active electrode circuitry contains amplifiers with active shielding techniques within the circuitry to shield external electrical radiation away from the sensitive circuitry.

In yet another aspect of the present invention, faraday cages surrounding the circuitry are included to reduce noise infiltration, such as 60 Hz power lines as seen in North America or 50 Hz in Europe and Asia for example, into the EEG signal.

In one preferred embodiment of the present invention, a micro-spike electrode, or/and conductive elastomer electrode are connected directly to active electrode circuitry which amplify the EEG signal at the source to reduce noise infiltration into the signal and can be mounted in a head-worn frame.

In one aspect of the present invention, said electrode(s) can be combined in a head-worn frame with the integration of visual, aural, or/and tactile feedback mechanisms.

In one preferred embodiment, the sensors are combined with a visual feedback mechanism such as electrochromic liquid crystal lenses that darken as a method of providing a penalty as feedback.

In another aspect of the present invention, a micro-spike or other type of electrode can be mounted on a bend-and-stay stainless steel wire which allows adjustability or formability of the placement of the arm to reach different areas of the cerebral cortex. The arm can be placed uni-laterally to provide placement options on either the left or right hemispheres of the brain.

In yet another aspect of the present invention, a conductive elastomer or other type of electrode can be placed on arms that can extend and retract in order to fit different head shapes and sizes.

In yet another aspect of the present invention, said extendable arms are attached the main lens frame with a hinge that allows opening and closing of the arms against the main lens frame body. Said hinge can be a traditional swing hinge of metal body or plastic body as seen in traditional eyeglass frames.

In one preferred embodiment of the present invention, said flexible living hinge can also be a “living-hinge” made of a rigid, semi-rigid, or flexible material. The flexible living hinge offers the advantage of minimal parts to assist in design, production, and assembly as well as inhibit propensity of breakage. In addition, the flexible living hinge improves the ability to over-extend the frame to an open position and articulation in both horizontal and vertical planes.

In one preferred embodiment of the present invention, said living hinge is comprised of a thermoplastic polyurethane (TPU) or other similar material that can be molded, extruded, or formed in other similar manner. In one aspect, the TPU material can be 3D printed.

In another preferred embodiment of the present invention, the TPU material can be 3D printed. Applying various methodologies, the 3D print process can be modified to increase or decrease elongation, flexibility, pressure, and the like to achieve desired feel and function.

Those skilled in the art can appreciate that the toolpath of the 3D print process can be modified to achieve various flexion properties of the TPU living hinge.

Sensors of various designs and materials have been used for the purpose of capturing and measuring physiologic signals. Previous technologies rely on low impedance connections to the body and such require cleaning and preparation of the skin which often involve abrading the skin and can often open risk to infection. These sensors often require the use of gels and pastes which can be messy and cumbersome.

Other sensor designs require moisture and are known as “wet electrodes” and often involve saline water or other electrolyte water to create a low impedance bridge to capture the physiologic signals.

Wet electrodes require preparation and are often disposable or one time use and can be messy, uncomfortable and difficult to work with.

In one aspect of the present invention a “micro-spike” electrode is designed as a high density of spikes within a small surface area to create a bed-of-nails effect to prevent the spikes from completely breaking through the skin but protrudes enough to reach through the first layers of the epidermis to achieve a low impedance connection to the body.

In one preferred embodiment of the present invention the micro-spike electrode can be made of any plastic or resin materials or of metallic or other conductive materials. In the case of plastic or 3D printed materials, the sensor can be coated with metallic substrate such as gold or silver to create a low impedance coating for contact with the body.

In one aspect of the present invention, the said micro-spike sensor can also be used as a stimulation electrode instead of measurement. In the case of stimulation, a signal such as signals seen in TENS units or TDCs is connected to a conductive part of the micro-spike electrode. The signals are transmitted through the conductive surface of the electrode which has a low surface resistivity (<1 ohm/cm2) and through the surface of the micro-spikes which are then transmitted through the epidermis.

In a different embodiment of the present invention a conductive elastomer is used in place of or in conjunction with the microspike, wet or traditional electrodes. The conductive elastomer offers the advantage of comfort, flexibility and versatility.

In one preferred embodiment of the present invention, said conductive elastomer can be made of silicone or other elastomeric material and contain materials such as but not limited to carbon, silver, gold, aluminum, nickel, graphite nickel plated graphite particles, or any combination thereof to increase the conductivity of the elastomeric substrate.

Those skilled in the art will appreciate the measures must be taken in the selection of material composition to reduce galvanization of the material which can occur when combining dissimilar metals. Galvanization can cause a high DC voltage offset which can disrupt the measurement of physiologic signals. One measure commonly implemented is the integration of chloride to reduce oxidation and galvanization. This is commonly seen in the case of silver-silver chloride electrodes in which the chloride component is added to reduce galvanization.

In another preferred embodiment of the present invention carbon, aluminum, silver, gold, or other metallic elements, in any combination can be used to achieve high surface conductivity. It should be noted that some materials have higher biocompatibility than other materials such as nickel or aluminum which certain people can have negative reactions to such as rashes or blisters. In this case, it is important to manage the ratio of the reactive materials within the compound and to take preventative measures in design of the sensors and manufacturing to limit the amount of the reactive materials on the surface and within the composite materials.

In one aspect of the present invention, said conductive elastomer sensor can be an o-profile extrusion of material which can then be cut and shaped with other mechanics to make contact with the body.

In one preferred embodiment of the present invention said O-profile extruded conductive elastomer tube is cut lengthwise to create a U or C shaped profile which can then be mounted in a frame. This offers the advantage of providing a slight spring or cushion to the material in order to provide enhanced comfort and contact with the body, and especially in contoured surfaces that are not flat such as the mastoid bone behind the ears.

In one aspect of the present invention the elastomeric electrodes are placed behind each ear and in contact with the skin around the mastoid bone which is a popular location for EEG reference measurements and ground electrode placements.

In yet another aspect of the present invention, a method of connecting a signal wire to the elastomeric material is achieved by attaching a metallic plate or clip to the end of the elastomeric material which than has the signal wire soldered or attached in some other manner to the metallic part. Because the elastomeric material has a low contact resistivity, only a small amount of surface area is required to achieve proper connection to said electrode.

In a different embodiment of the present invention, the micro-spike electrode, or/and conductive elastomer electrode can be mounted in headworn frame which contains electronics for the purpose of amplification of the physiologic signals.

In one aspect of the present invention, said electrode(s) can be combined in a headworn frame with the integration of visual, aural, or/and tactile feedback mechanisms.

In one preferred embodiment, said sensors are combined with a visual feedback mechanism such as electrochromic LCD lenses that darken as a method of providing a penalty as feedback.

In another aspect of the present invention, said micro-spike or other type of electrode can be mounted on a bend-and-stay stainless steel wire which allows adjustability or formability of the placement of the arm to reach different areas of the cerebral cortex. The arm can be placed unilaterally to provide placement options on either the left or right hemispheres of the brain. The illustrations in the drawing depict the left side model in which the top sensor arm comes out of the left arm and said top sensor can reach anywhere along the centerline of the cortex as well as any of the traditional site placements on the left hemisphere of the cerebral cortex. A right side model (not illustrated in the drawings) also exist which is symmetrically mirrored to the left side model, with the top sensor arm coming from the right arm and reaching anywhere down the centerline or on the right hemisphere of the cerebral cortex. In the case of the left hand model, the reference sensor is normally tied to left mastoid and ground is tied to right mastoid, and in the case of the right hand model, the reference sensor is normally tied to right mastoid and ground is tied to left mastoid. However, these locations can be reversed such that the right hand model has the top sensor coming from the right arm and reference is tied to left mastoid and ground is tied to right mastoid and visa-versa for the left hand model. Alternative embodiments of the sensor orientation may be applied, such as two or more active channels, without departing from the scope of the present invention.

In one preferred embodiment of the present invention, the top sensor arm can be bent and formed by hand to position the top sensor in different locations on the cerebral cortex.

In a different aspect of the present invention, embodiment of the present invention, the EEG electrode which can be micro-spike or other type of electrode capable of measurement of physiologic signals is coupled with infrared light emitting diodes (LEDs) to provide photobiostimulation (or photostimulation) to the area of measurement.

In another aspect of the present invention, the infrared therapy can be transmitted via a laser optical unit and carried through a fiber optic cable with a terminating lens output in contact with or near the scalp area of measurement in respect to the electrode.

Those skilled in the art will appreciate that photobiostimulation or photobiomodulation is a novel noninvasive method used to promote neuroprotection and repair of injured neuronal pathways by activating endogenous mechanisms that are involved in both processes. It is thought that the combination of photobiostimulation and biofeedback or neurofeedback can enhance the effectiveness of the therapy.

In a different aspect of the present invention, the measurement electrode head in contact with the body is combined with infrared transmitting LEDs with a photonic output between 600 nm and 900 nm wavelength and intensity of between (7.5 mW/cm2 and 75 mW/cm2). It is appreciated that various wavelengths can reach various depths of neural tissue in the cerebral cortex and that various intensities can increase or decrease the effectiveness of the therapy. It is also appreciated that an intensity output greater than 75 mW/cm2 may cause overheating of the tissue or other damage that can affect the integrity of the neural cell structure and may result in cell death or impairment of normal functioning.

In yet another aspect of the present invention, the conductive elastomer or other type of electrode can be placed on arms that can extend and retract in order to fit different head shapes and sizes.

In yet another aspect of the present invention, the said extendable arms are attached the main lens frame with a hinge that allows opening and closing of the arms against the main lens frame body. Said hinge can be a traditional swing hinge metal body or plastic body as seen in traditional eyeglass frames. Said hinge can also be a “living-hinge” made of a rigid, semi-rigid, or flexible material. Said living hinge offers the advantage of minimal parts to assist in assembly and inhibit breakage.

In one preferred embodiment of the present invention, the living hinge is used and comprised of a thermoplastic polyurethane (TPU) or other similar material that can be molded, extruded, or formed in other similar manner. In one aspect, the TPU material can be 3D printed.

In another preferred embodiment of the present invention, the TPU material can be 3D printed and in various ways the 3D print process can be modified to increase or decrease elongation, flexibility, pressure, and the like to achieve desired feel and function.

Those skilled in the art can appreciate that the toolpath of the 3D print process can be modified to achieve various flexion properties of the TPU living hinge.

### BRIEF DESCRIPTION OF THE DRAWINGS

FIG. 1 is an image of the neurofeedback system with integrated active dry electrodes and electrochromic lens feedback.

FIG. 1A is an image of the same system illustrated in FIG. 1 with the top sensor arm in frontal lobe orientation.

FIG. 1B is an image of the same system illustrated in FIG. 1 with the top sensor arm in occipital lobe orientation.

FIG. 2 is the same system shown in FIG. 1 in a closed position.

FIG. 3 is a detailed view of the main connection points around the flexible living hinge of said system.

FIG. 4 is a detailed view of the top sensor holder connection points.

FIG. 5 is the same top sensor holder as illustrated in FIG. 4 represented as a digital CAD model.

FIG. 6 is an inside view of the part illustrated in FIG. 4 and FIG. 5 illustrated as a digital CAD model.

FIG. 7 is an image of the inside view of the top sensor holder illustrated in FIGS. 4, 5, and 6.

FIG. 8 is an image of the active electrode PCB and dritz snap connector which is mounted in the top sensor holder.

FIG. 9 is in image of the top sensor that is snapped into dritz snap connector in FIG. 8.

FIG. 10 is an image of the left side arm in extended position of the said system.

FIG. 11 is an image of the left side arm in closed position.

FIG. 12 is an image of the left ear sensor holder and active electrode PCB.

FIG. 13 is an image of the ear sensor holder without PCB.

FIG. 14 is an image of the ear sensor.

FIG. 15 is an image of ear sensor frame without the sensor material.

FIG. 16 is an image of the ear sensor material flattened down.

FIG. 17 is an image of the metal clip that is attached to the ear sensor holder material.

FIGS. 18 and 19 are images of the flexible living hinge.

FIG. 20 is an image of the connection points between main frame, hinge, and side arm. FIG. 21 is an image of the main frame and electrochromic lenses.

FIG. 22 is an image of the main frame with electrochromic lenses inserted.

FIG. 23 is an image of the system with arms opened to full horizontal articulation and a detailed close view of the right hinge in fully opened position.

FIG. 24 is an image of the system with arms twisted to full vertical articulation of the arms.

FIG. 25, FIG. 26, FIG. 27, and FIG. 28 are illustrations of various 3D printing toolpaths for the flexible living hinge illustrated in FIG. 18 and FIG. 19.

FIG. 29, FIG. 30, and FIG. 31 are engineering drawings illustrating the dimensions of micro-spike sensing/stimulating probe.

FIG. 32 is an image of the micro-spike sensing/stimulating probe illustrated in FIG. 29, FIG. 30, and FIG. 31.

FIG. 33 is an alternative headset design containing said micro-spike sensing/stimulating probe.

FIG. 34 is an isometric view of O-profile extrusion tubing with dimensions.

FIG. 35 is a chart representing physical properties of the tubing of FIG. 34.

FIG. 36 is a table illustrating physical properties of a TPU material of the flexible living hinge.

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

The following embodiments depict a system which obtain physiologic data through dry non-contact active EEG sensors which have been implemented for the creation of the illustrated prototypes in the drawings. Let it be understood that additional implementations are possible, such as, by example but not limited to, the use of near-infrared spectroscopy for the purpose of tracking neural activity instead of EEG, without departing from the scope of the present invention.

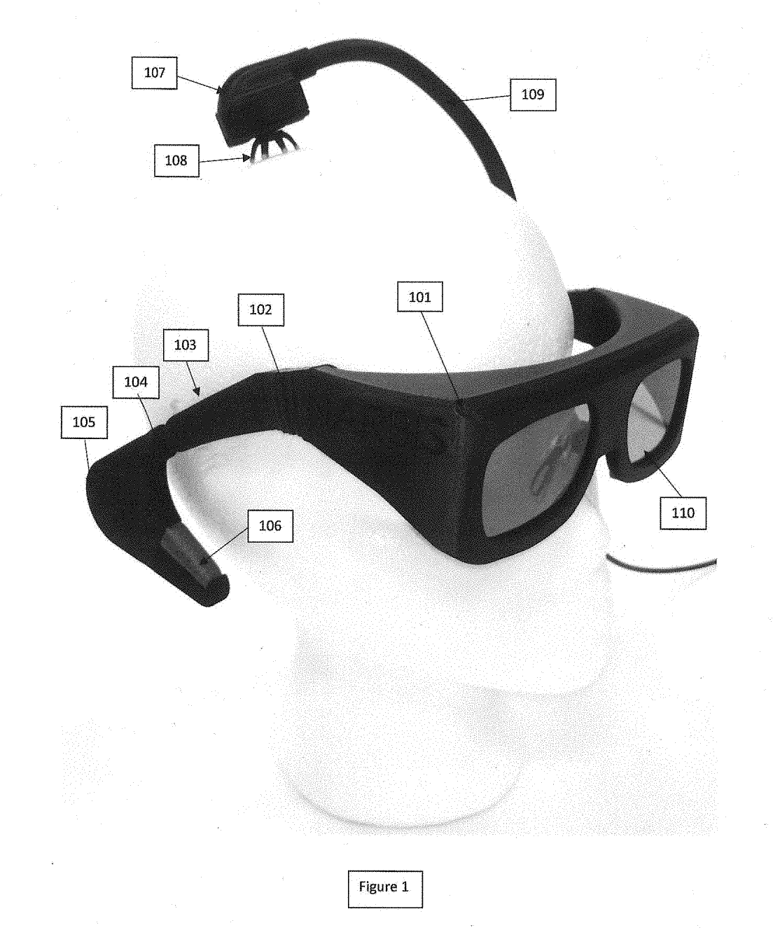

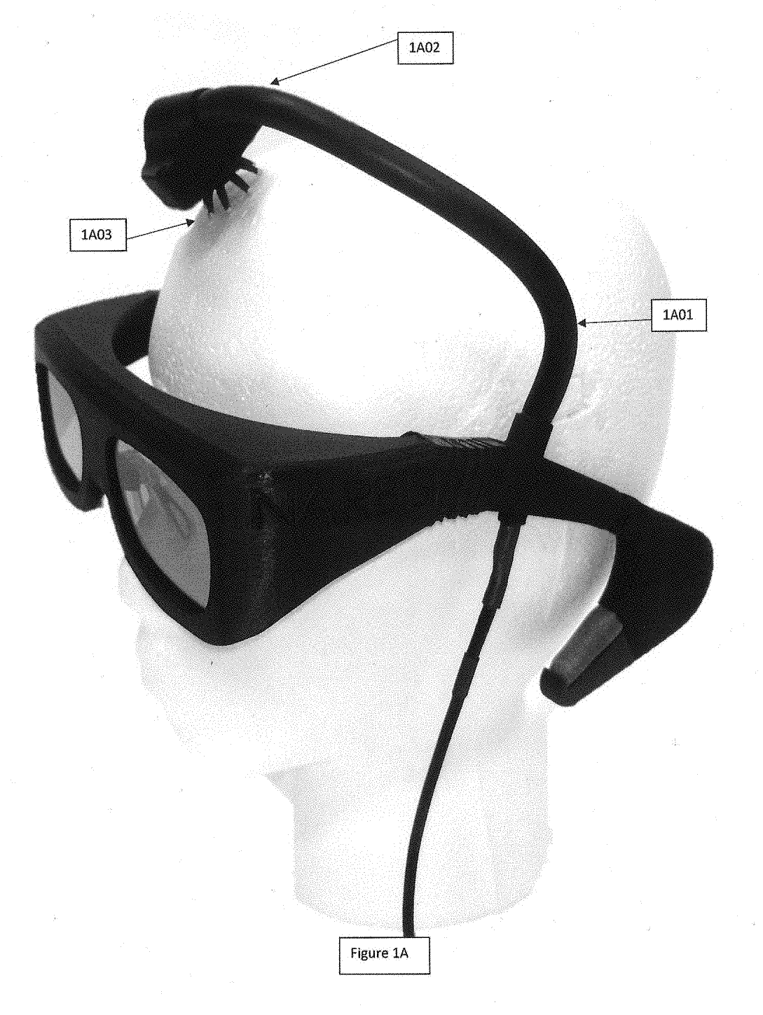

FIG. 1 is an image of the neurofeedback system with integrated active dry electrodes and electrochromic lens feedback. Main frame 101 contains the electrochromic lenses 110. Main frame 101 is attached to hinge 102 which is attached to side arm 103. Side arm 103 is attached to slider arm 104 which is attached to side (or ear) sensor holder 105 which contains ear sensor

106. Top sensor arm 109 is attached top sensor holder 107 which is connected to top sensor 108.

In one preferred embodiment of the present invention, the top sensor arm can be bent and formed by hand to position the top sensor in different locations on the cerebral cortex. The illustrations that follow in FIG. 1A and FIG. 1B are exemplary of possible site placement for the top sensor and that alternative orientations are possible, such as a 180 degree bend in the wire to place the top sensor at T9, T7, FT9, FT7, TP9, TP7, and including but not limited to P9, P7, F9, F7, AF7, AF3, Fp1, PO7, P03, 01, F3, F1, FC3, FC1, FC2, and so on. The placements would be mirrored on the right hemisphere with the right hand model version.

FIG. 1A is an image of the same system illustrated in FIG. 1 with the top sensor arm in frontal lobe orientation. Top sensor arm is bent forwards at location 1A01 and bent again at location 1A02 such that top sensor is resting on the frontal lobe, at location 1A03, which is near Fpz of 10-20 site placement diagram.

FIG. 1B is an image of the same system illustrated in FIG. 1 with the top sensor arm in occipital lobe orientation. Tope sensor arm is bent backwards at location 1B01 and bent again at location 1B02 such that tope sensor is resting on the occipital lobe, at location 1B03, which is near Oz of 10-20 site placement diagram.

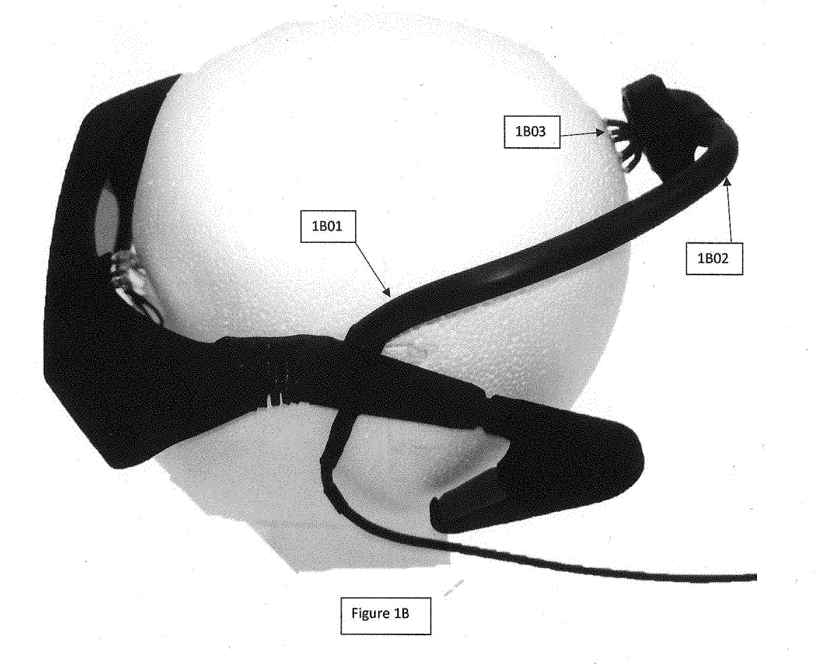

FIG. 2 is the same system shown in FIG. 1 in a closed position. Cable 113 is connected to an external ADC and processor. The nose piece 111 is made of clear silicone and has semi-rigid metal core that can be opened or closed to different widths for positional adjustment and comfort. Hinge 112 is in closed position.

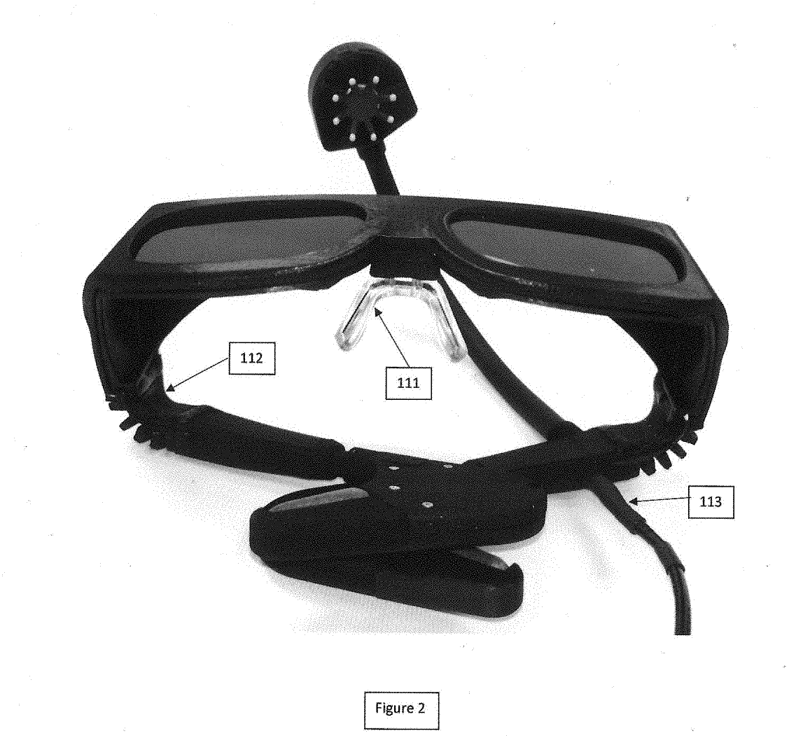

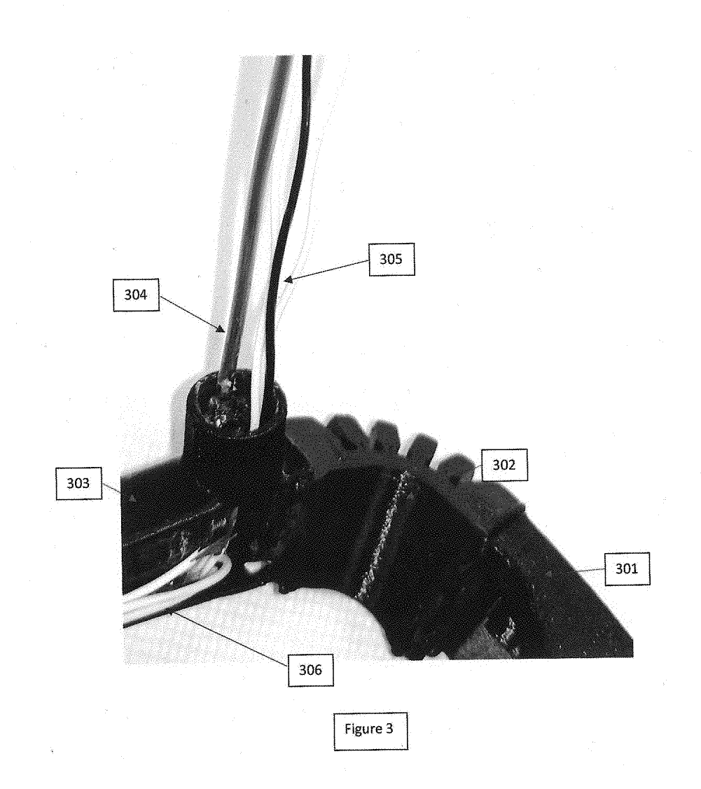

FIG. 3 is a detailed view of the main connection points around the flexible living hinge of said system. Main frame 301 is shown which is heat-fused as way of attachment to hinge 302. Hinge 302 is heat-fused to side arm 303. Wires 306 connect to ear sensor. Top sensor arm 304 is made of stainless steel bend-and-stay wire. Top sensor wire 305 are shown next to top sensor arm 304.

FIG. 4 is a detailed image of the top sensor holder connection points. Top sensor arm 401 is shown attached at connection point 404 to top sensor holder 403. Top sensor wires 402 are shown inserted into top sensor holder 403.

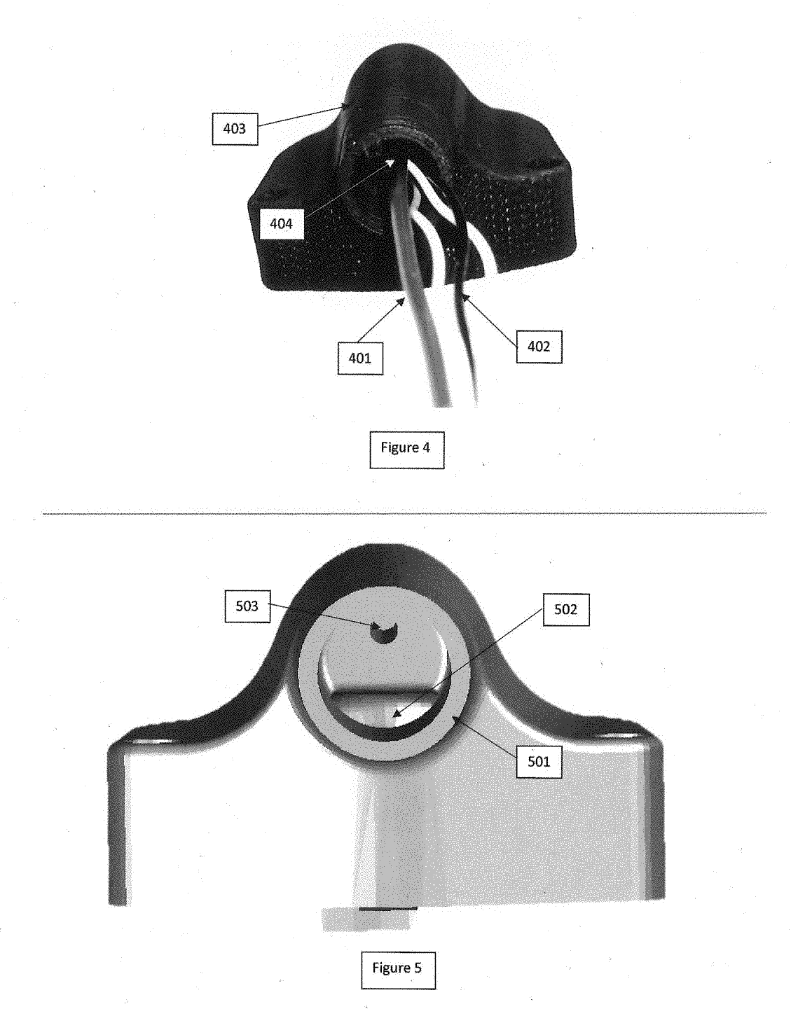

FIG. 5 is the same top sensor holder as illustrated in FIG. 4 represented as a digital CAD model. The boss 501 holds the black EPDM rubber tube which houses the stainless wire and electrical wires connecting the top sensor holder to the side arm of the assembly. The wire hole 502 allows the electrical wires to connect to the top sensor PCB. The wire bore 503 allows the stainless wire 401 in FIG. 4 to be attached to the top sensor holder.

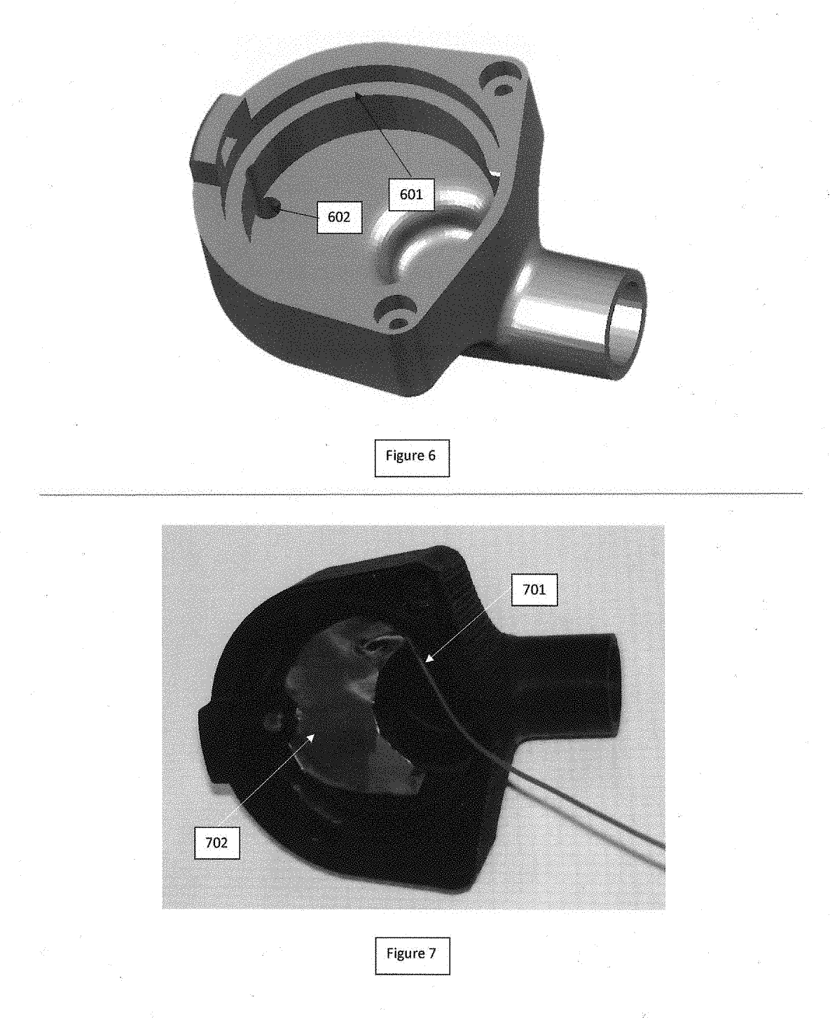

FIG. 6 is an inside view of the part illustrated in FIG. 4 and FIG. 5 illustrated as a digital CAD model. The light pipe hole 602 allows a plastic light pipe to be inserted to transmit light from an LED on the top sensor PCB to the outside of the case. The light could be used to indicate power on or poor vs good signal quality.

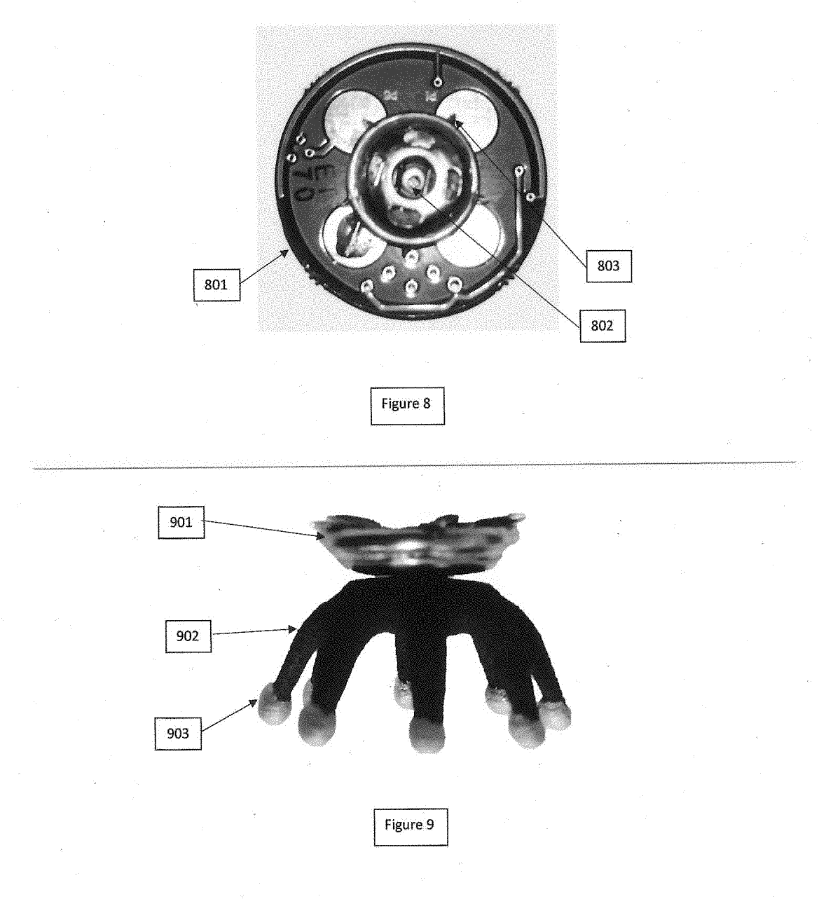

FIG. 7 is an image of the inside view of the top sensor holder illustrated in FIGS. 4, 5, and 6. Copper faraday cage 702 is soldered to ground wire 701 and covered with kapton tape to prevent short between the faraday cage and the PCB. The faraday cage effectively shields noise such as 50 Hz/60 Hz interference from electrical systems and components as well as other radio frequency or electromagnetic radiation. The ground wire 701 is attached to the PCB ground.

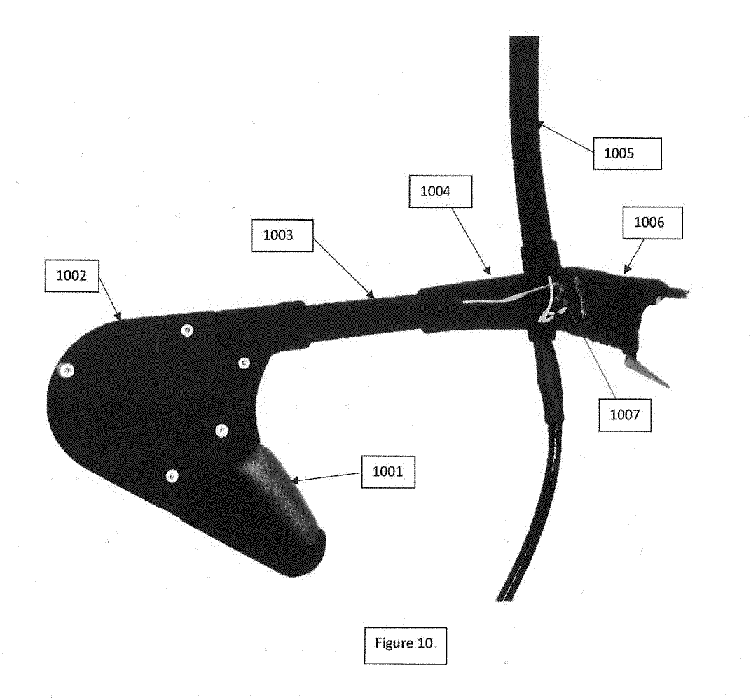

FIG. 8 is an image of the top sensor holder PCB and dritz snap connector which is mounted in the top sensor holder. Top sensor PCB 801 is soldered to PCB pads 803. The dritz snap button 802 is used to connect to the top sensor.

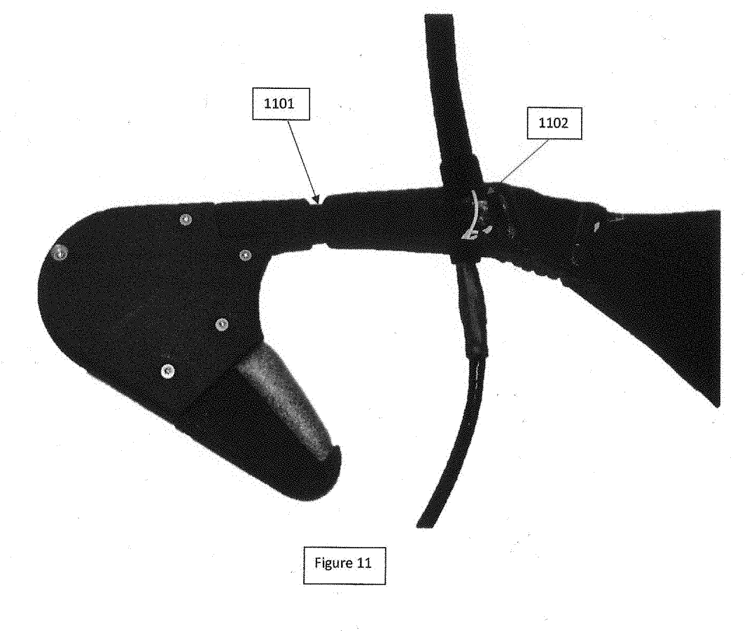

FIG. 9 is an image of the top sensor that is snapped into dritz snap connector on PCB in FIG. 8. dritz snap button 901 is attached to top sensor 902. The black fingers are flexible outwards. Top sensor tips 903 are electrically conductive and carry biosignals towards the dritz snap button connection point to the dritz snap button 901, further to top sensor PCB pads 803 and top sensor PCB 801 in FIG.8.

FIG. 10 is an image of the left side arm in extended position of the said system. Silicone ear sensor 1001 is made of a silicone composite material which contains metallic components such as carbon, nickel, or the like to make the material conductive to create a low-impedance connection between the amplifier and the body. Silicone ear sensor 1001 is positioned inside ear sensor holder 1002 which also contains the ear sensor PCB. Ear sensor holder 1002 is attached to ear slider arm 1003 which slides in and out of side arm 1004. The wires connected to the ear sensor PCB inside of ear sensor holder 1002 is anchored by way of adhesive or mounting clip inside side arm 1004 at attachment point 1007. Positioning the wire anchor at attachment point 1007 allows the wires to slide in and out of ear slider arm 1003. The wires coming from side arm 1004 going to top sensor holder are positioned alongside stainless wire inside EPDM black rubber tube 1005. The side arm 1004 is attached to rubber hinge 1006.

Referring still to FIG. 10, those skilled in the art will appreciate that side arm 1004 is made of a rigid material such as ABS or ASA and rubber hinge 1006 is made of a flexible material such as a thermoplastic polyurethane (TPU) and there is difficulty in finding an adhesive that is compatible with both materials. In this case, heat-fusion or welding was used to attach hinge 1006 to side arm 1004 to create a permanent bond. The outside wall of hinge 1006 is heated using controlled hot air to ˜350 degrees Celsius, until the material is slightly molten, then pressed against side arm 1004 until it cools back to room temperature.

Those skilled in the art will appreciate that the heat fusion procedure adopted to permanently adhere the rigid ASA or ABS materials to the flexible TPU materials described above was implemented for simplicity of building prototypes and alternative methodologies are available such as over-molding the flexible parts on top of the rigid parts in an injection molding process.

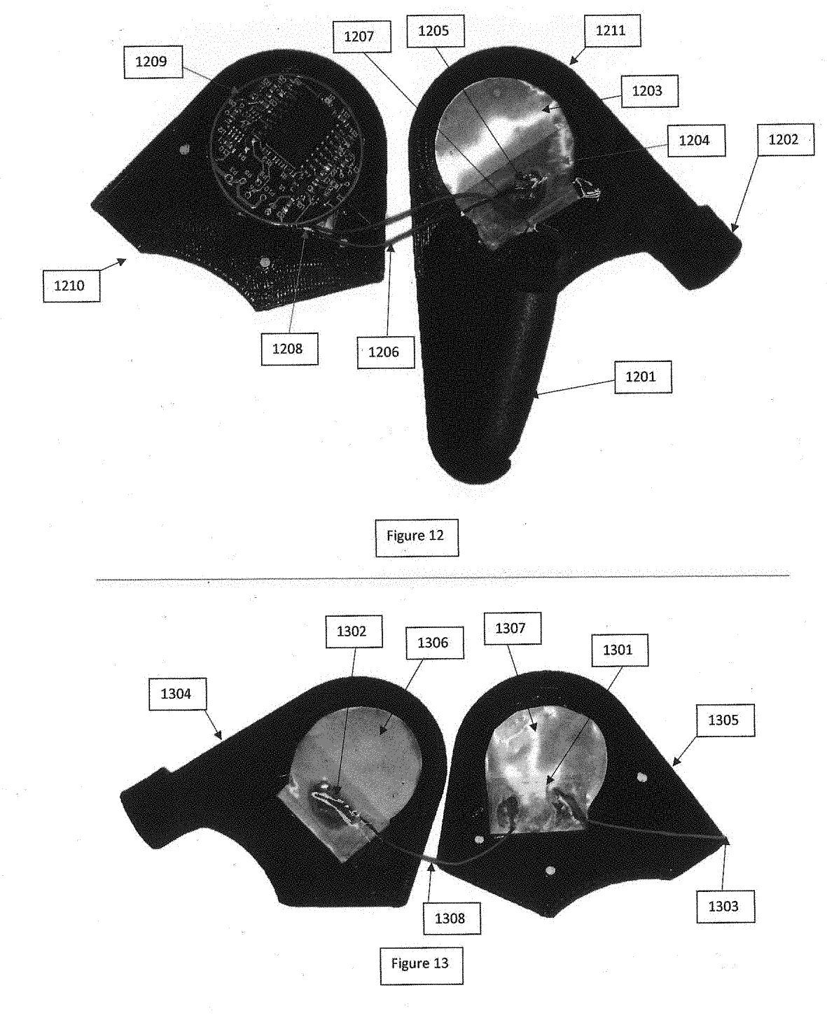

FIG. 11 is an image of the left side arm in closed position. Slider arm 1101 is shown in compressed or closed position with the end of slider arm 1101 pressed against the inside wall of side arm at location 1102.

FIG. 12 is an image of the left ear sensor holder and active electrode PCB. Ear sensor holder 1002 of FIG. 10 is attached to slider arm 1003 of FIG. 10 at attachment point 1202 the ear sensor wires pass through both slider arm 1003 of FIG. 10 and ear sensor holder attachment point 1202 of FIG. 12 and connected finally to ear sensor PCB 1209. A faraday cage bottom 1208 is used to enclose the ear sensor PCB 1209 inside of ear sensor cover 1210 and the other side of the faraday cage top 1203 inside ear sensor holder 1211.

Referring still to FIG. 12, Let it be known that various implementations of the faraday cage are possible and that the implementation of the faraday cage described have been adopted for simplicity and ease of prototype assembly. It is understood that radio frequency radiation may be leaking through the faraday cage bottom 1208 and faraday cage top 1203 and that a preferred implementation of the faraday cage would be a design the completely encloses the PCB without any holes or gaps.

Referring still to FIG. 12, faraday cage bottom 1208 and faraday cage top 1203 are made of 5 mil annealed copper alloy 110, 99.9% pure copper. Blue wire 1206 is soldered to faraday cage bottom 1208 and faraday cage top 1203 at point solder point 1205 to create electrical continuity between the two parts of the faraday cage. Red wire 1207 is connected to silicone ear sensor 1201 and ear sensor PCB 1209.

FIG. 13 is an image of the ear sensor holder without PCB. Ear sensor holder 1304 is shown with faraday cage top 1306 and solder point 1302 which connects blue wire 1308 to bottom faraday cage 1307 at solder point 1301 inside ear sensor cover 1305. Note that bottom parts of faraday cages have been covered with kapton tape at the solder points to protect the copper and solder points from making contact with the ear sensor PCB and sensor to prevent shorting of the circuit. Finally blue wire 1303 is used to make electrical continuity from the faraday cage to the ear sensor PCB ground circuit.

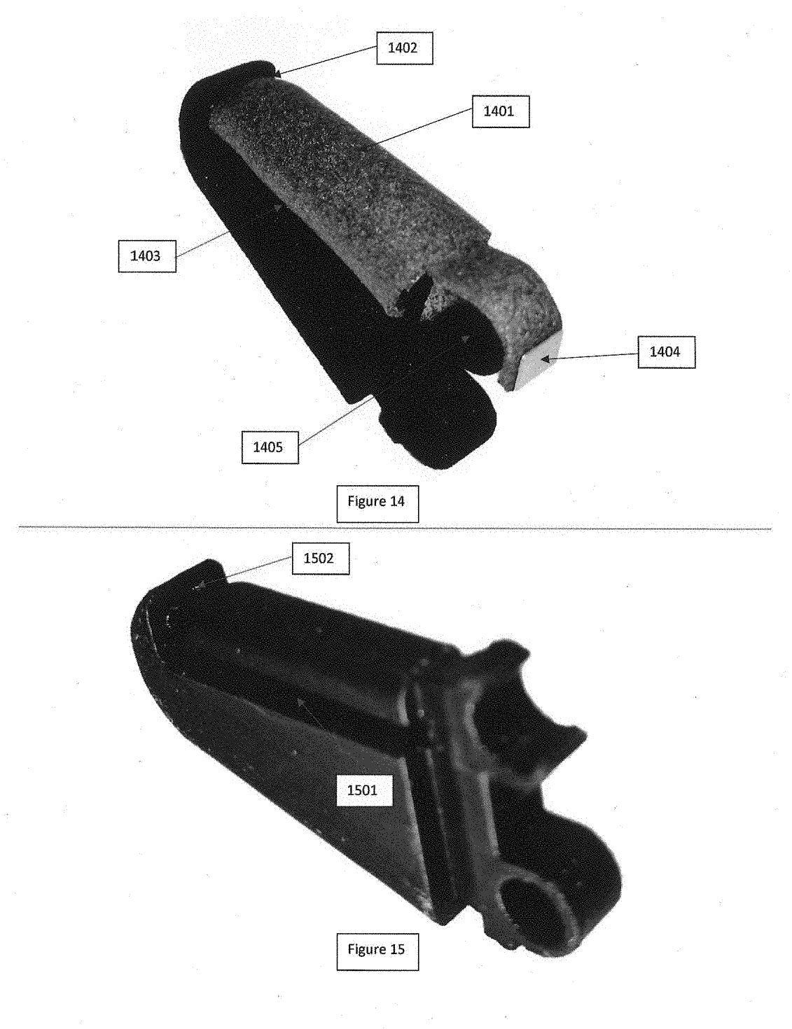

FIG. 14 is an image of the ear sensor. Silicone ear sensor 1401 is trimmed as positioned inside of ear sensor lobe side slot 1403 and ear sensor lobe top slot 1402 and wrapped around mounting hole 1405. A metal wire clip 1404 is placed on the tail of the silicone ear sensor 1401 which is later used to solder a wire to, creating electrical continuity from the silicone ear sensor 1401 to ear sensor PCB (not shown is this FIG. 14.).

FIG. 15 is an image of ear sensor frame without the sensor material. Ear sensor top slot 1502 and ear sensor side slot 1501 are shown, which is an appropriate thickness to allow the silicone ear sensor material to be glued in the slots.

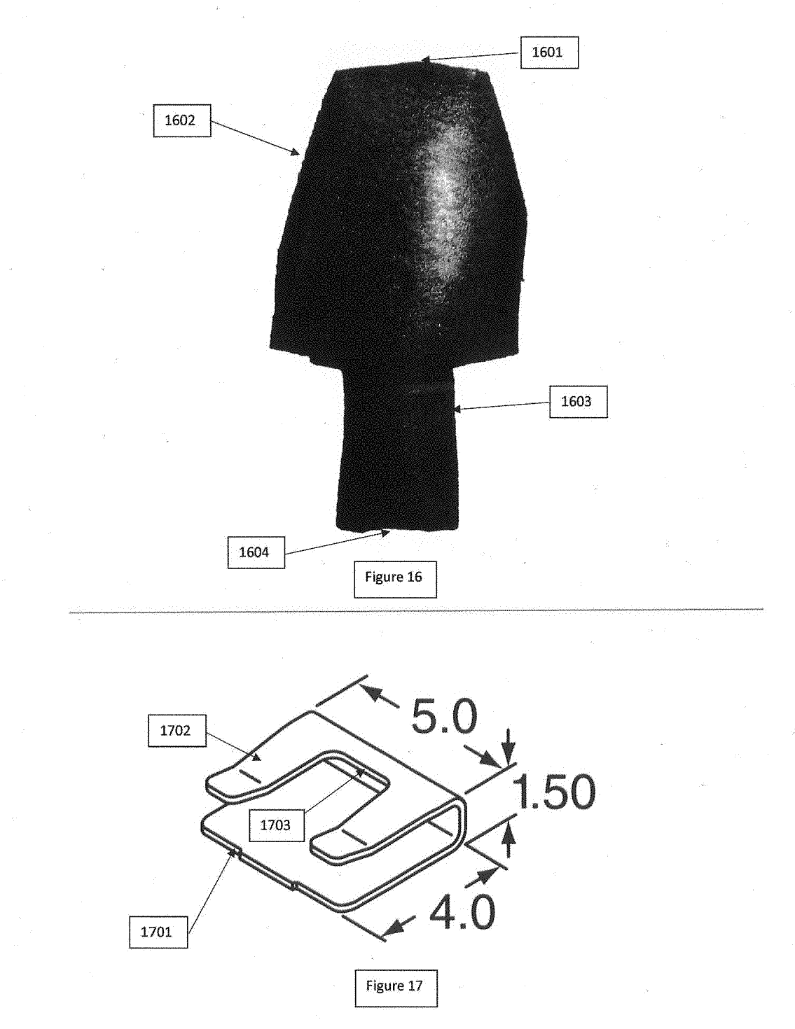

FIG. 16 is an image of the ear sensor material flattened down. Ear sensor material top 1601 of FIG. 16 is placed inside ear sensor lobe top slot 1502 of FIG. 15 and Ear sensor material side 1602 is placed inside ear sensor lobe side slot 1501 of FIG. 15. The ear sensor material tail 1603 is trimmed and ear sensor wire clip is placed on the end of tail 1603 at mounting location 1604.

FIG. 17 is an image of the metal clip that is attached to the ear sensor holder material. The ear sensor wire clip is shown in FIG. 17 which is clipped onto the end of ear sensor tail 1603 of FIG. 16 at mounting location 1604 of FIG. 16. The sensor wire clip bottom side 1701 is used to solder the ear sensor PCB red wire 1207 of FIG. 12. to create electrical continuity between the sensor material and the PCB. Wire clip inside wall 1703 is pressed all the way against the end of the tail at mounting location 1604 of FIG. 16 and wire clip fingers 1702 and inside of wire clip bottom side 1701 are glued onto silicone sensor material to create a permanent bond.

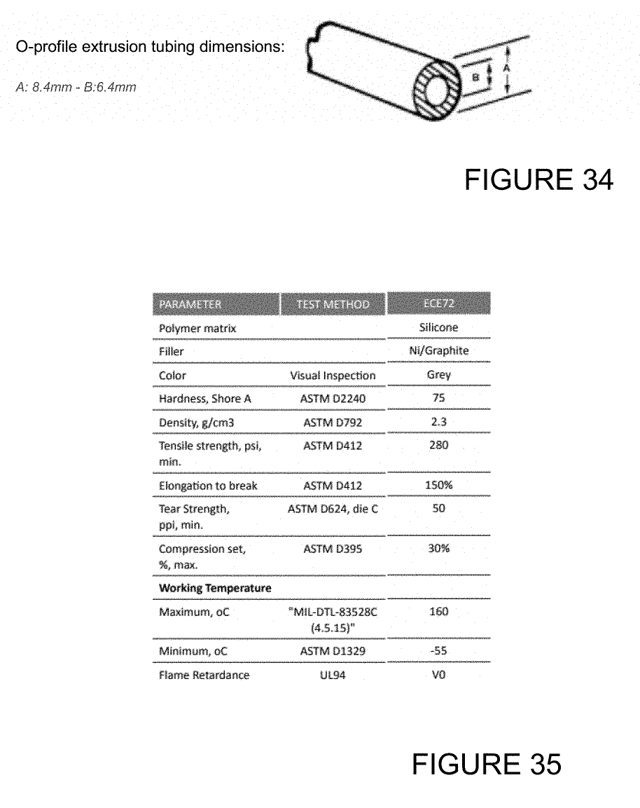

Let it be known that different implementations of the silicone ear sensor material are possible. The implementation illustrated here is a preferred method that allows the use of off-the-shelf materials and can be made without tooling. In the implementation illustrated above, an off-the-shelf conductive elastomeric tubing is used with an O-profile extrusion with an inside diameter of 6.4 millimeters and outside diameter of 8.4 millimeters, as illustrated in FIG. 34. The O-profile tubing is then split lengthwise down the center, and then trimmed as shown in FIG. 16.

In one preferred embodiment of the present invention, O-profile extrusion tubing is used which contains Nickel and Graphite fillers to create electrical conductivity of the material. FIG. 35 is a chart that represents the physical properties of the O-profile extrusion tubing.

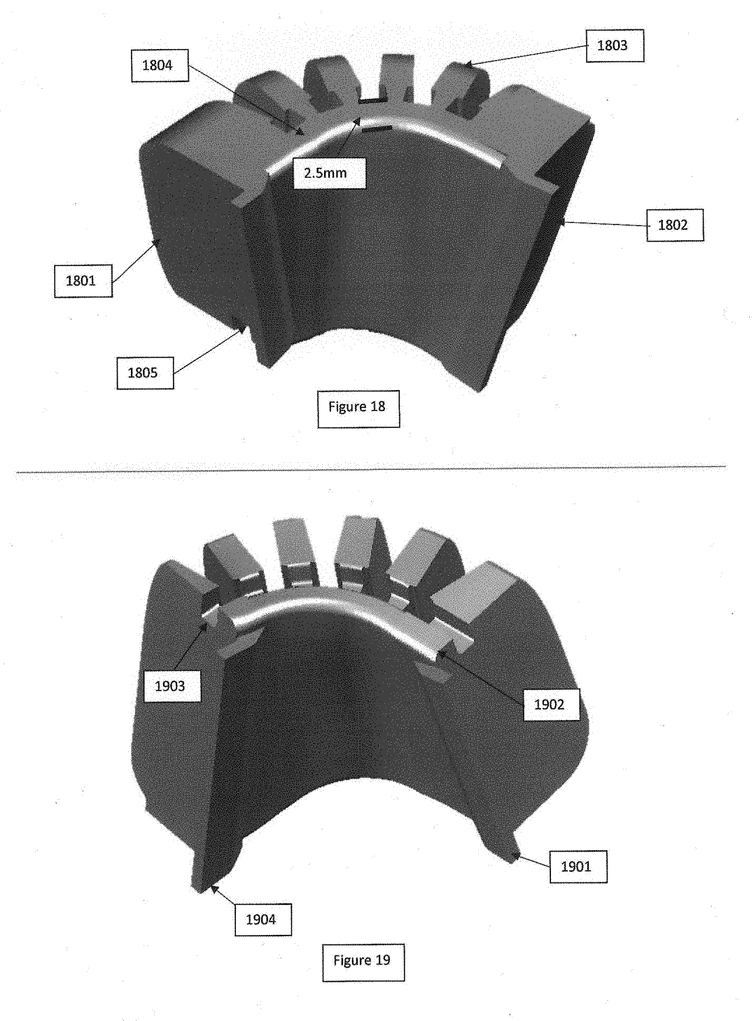

FIGS. 18 and 19 are images of the flexible living hinge. As described earlier, said flexible living hinge is made from a thermoplastic polyurethane (TPU) but could also be made from other materials with similar properties as well.

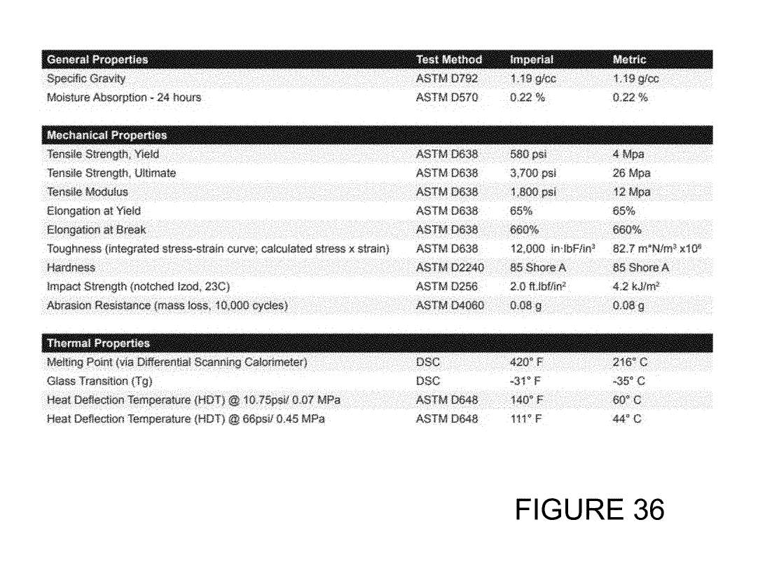

In one preferred implementation of the current invention, a TPU material of 85 Shore A hardness is used which exhibits the physical properties illustrated in FIG. 36.

Hinge back mating wall 1801 is attached to side arm 103 of FIG. 1 and hinge front mating wall 1802 is attached to main frame 101 of FIG. 1 using the heat-fusion method described earlier to bond the hinge TPU material to the rigid ASA/ABS material of side arm and main frame. Wire channel 1805 is placed at the bottom of the hinge to allow a guide for wire to be mounted and held in place.

Referring still to FIG. 18, hinge ribs 1803 are added to the flexible living hinge to create structural integrity with hinge in the open position. The hinge ribs 1803 also limit how far the hinge opens. The hinge ribs 1803 can be made more narrow (or one or more of the ribs removed) which would allow the hinge to open farther, or hinge ribs 1803 can be made wider (or more ribs added) which would all the hinge to open less.

Referring still to FIG. 18, hinge inner wall 1804 thickness defines the resistance of the hinge opening and closing. By increasing thickness hinge inner wall 1804, the hinge becomes more rigid and requires more force to open, and in the open position, a thicker inner wall create more force causing the hinge to close. By decreasing the thickness of hinge inner wall 1804, this will make the hinge less rigid and allow it to open more freely (or with less resistance, using less force to open). A thinner hinge inner wall 1804 also creates less closing force than a thicker wall.

In one preferred implementation of the current invention, a wall thickness of 2.5 mm produces the desired characteristics of hinge performance for this application. The hinges are 3D printed using TPU material in the closed position to achieve the desired closing behavior. The closing behavior of the hinges, when placed in the frame in accordance of what is described above, creates a gentle compression around the head. The gentle compression around the head keeps the headset in place, without causing physical discomfort, which also reduces movement, and therefore, reducing motion artifact in the EEG signal.

In another preferred embodiment of the present invention, said 3D printed TPU flexible living hinges are manufactured with various toolpaths to achieve different flexion and compression behaviors of the opening and closing articulations of said hinge. Those skilled in the art will appreciate that by varying the orientation of the toolpath, different characteristic of mechanical hinge behavior may be achieved.

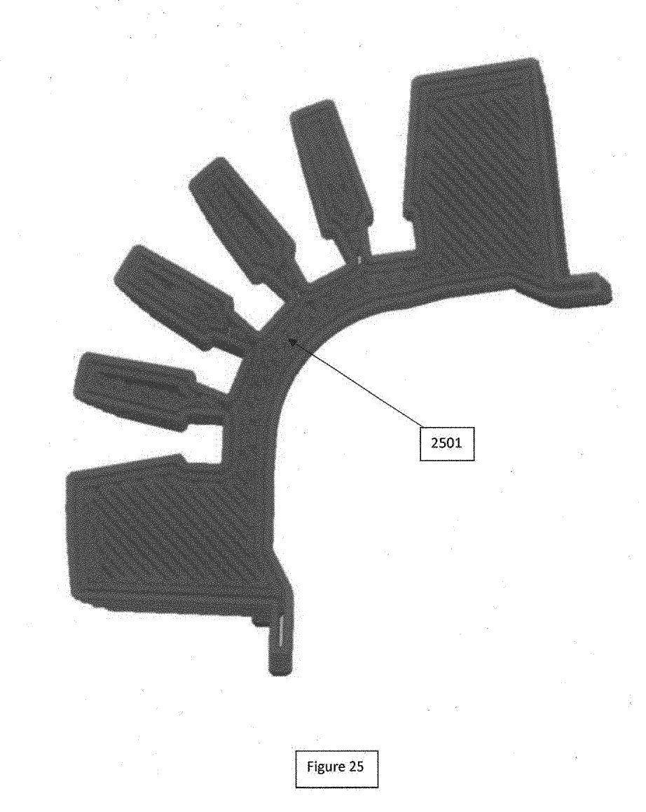

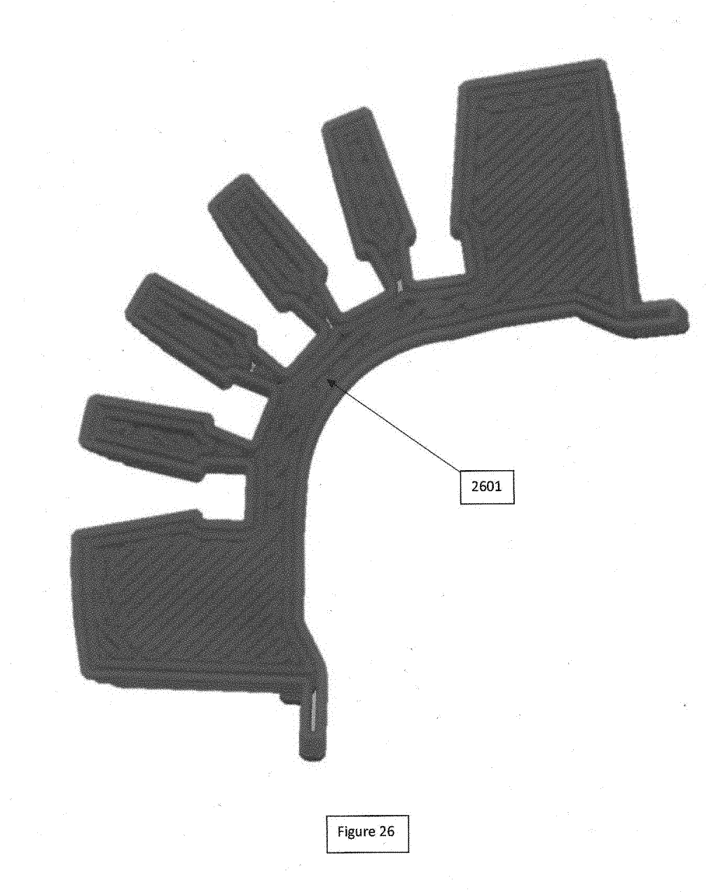

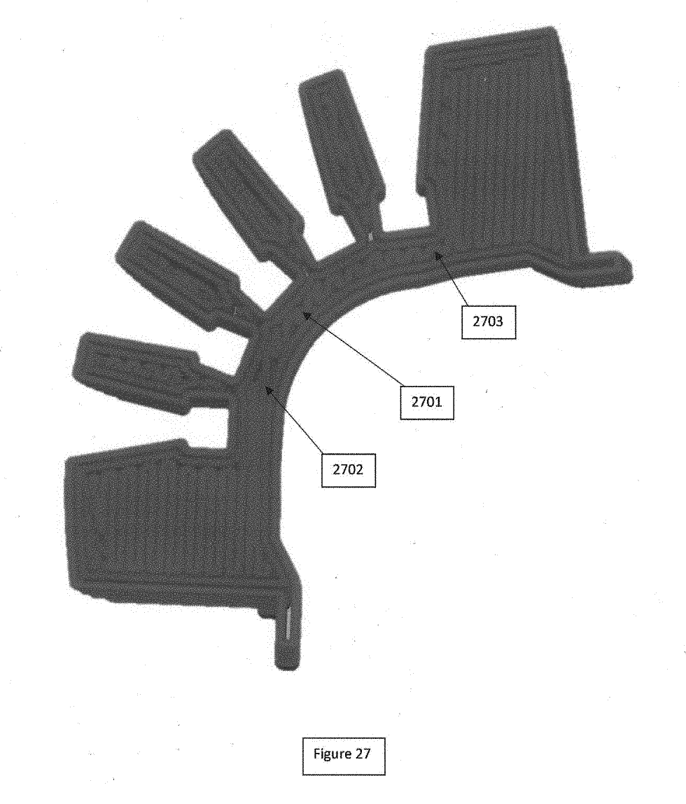

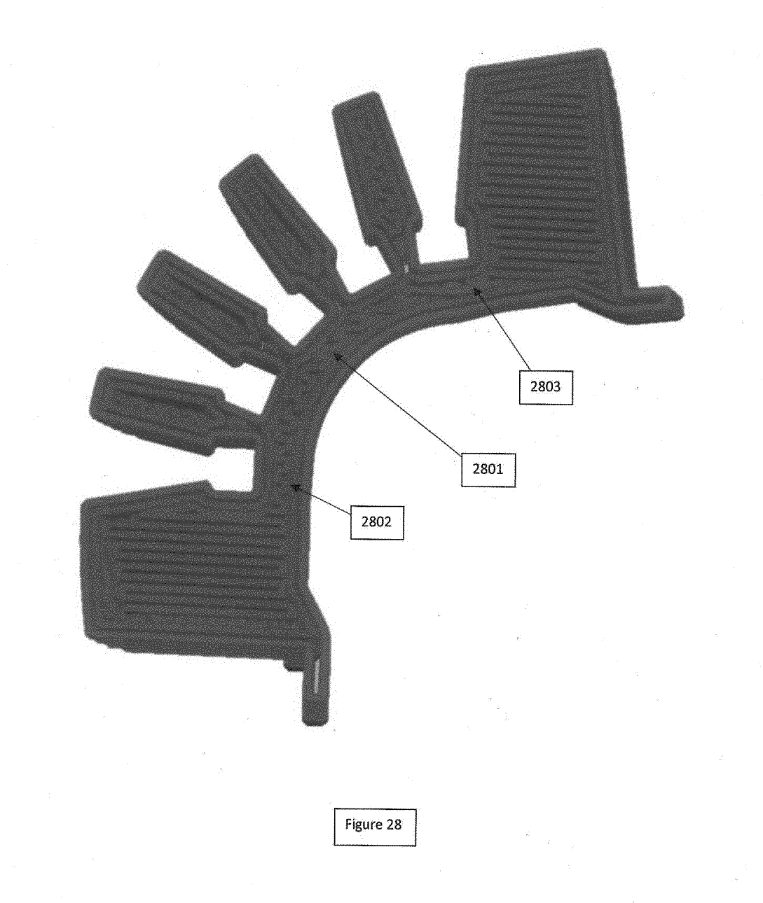

Referring now to FIG. 25, the 3D print toolpath for said flexible living hinge is illustrated with a 45 degree angle coming diagonally from bottom right of the part to the top left. The toolpath 2501 is perpendicular to the vertex of the arch in the center of the hinge. This orientation would create the least resistance in hinge closure.

Referring now to FIG. 26, the 3D print toolpath for said hinge is illustrated at a negative 45 degree angle coming diagonally from the bottom left to top right. The toolpath 2601 is parallel to the vertex of the arch in the center of the hinge. This orientation would create the greatest resistance in hinge closure.

Referring now to FIG. 27, the 3D print toolpath for said hinge is illustrated at a positive 90 degree angle coming diagonally from the bottom left to top right. The toolpath 2701 is at a positive 45 degree angle to the vertex of the arch in the center of the hinge. Toolpath at location 2702 is nearly parallel hinge inside wall which would create least resistance at location 2702. At location 2703, the toolpath is nearly perpendicular to hinge inside wall which would create the greatest resistance at location 2703.

Referring now to FIG. 28, the 3D print toolpath for said hinge is illustrated at a negative 90 degree angle coming diagonally from the bottom left to top right. The toolpath 2801 is at a negative 45 degree angle to the vertex of the arch in the center of the hinge. Toolpath at location 2803 is nearly parallel hinge inside wall which would create least resistance at location 2803. At location 2802, the toolpath is nearly parallel to hinge inside wall which would create the greatest resistance at location 2802.

The aforementioned variations in toolpaths allows for different parts of the hinge to behave differently without modifying the exterior dimensions of said hinge. For example, one side of hinge is held more rigid, while allowing the opposite side of the hinge to be held less rigid. A similar control of behavior can be achieved by varying the wall thickness of the inside vertex of the hinge but this requires modifying the dimensions of said hinge which could be less desirable in certain applications.

Referring now to FIG. 19, the flexible living hinge shown in FIG. 18 is shown upside down. Hinge flaps 1901 and 1904 are shown which wrap around the mating surfaces to create extra reinforcement and also create a cleaner look to the design. Hinge inner wall 1902 is shown next to wire channel 1903.

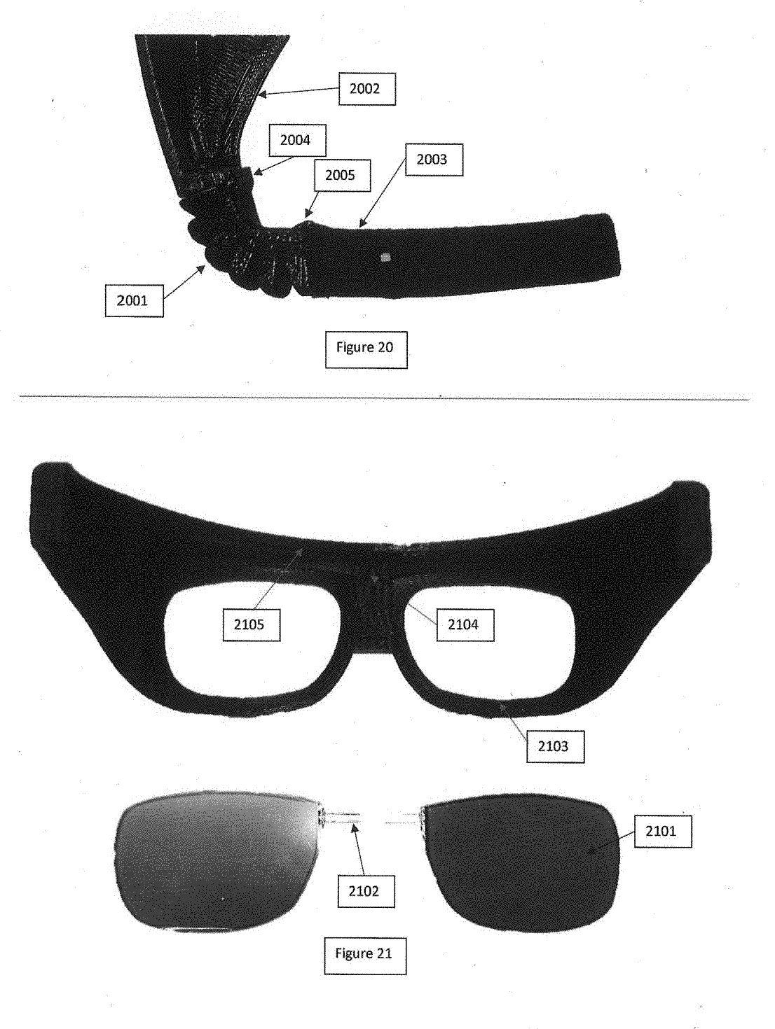

FIG. 20 is an image of the connection points between main frame, hinge, and side arm. Main frame 2002 is shown attached to flexible living hinge 2001. The hinge flaps 1904 of FIG. 19 is shown as hinge flap 2004 in FIG. 20. The hinge flap 2004 is covering part of main frame 2002 for bond strength reinforcement which prevents the mating surfaces from separation when hinge is opened and stress is applied outwards, away from the closed hinge position. Hinge flap 2005 is shown covering part of side arm 2003.

FIG. 21 is an image of the main frame and electrochromic lenses. Electrochromic lens 2101 is shown with attached lens connector pins 2102. One of the lens connector pins 2102 is positive polarity and the other pin is negative polarity. The lens connector pins 2102 are soldered to a coaxial wire with the signal wire on the inside of the coaxial cable being connected to positive polarity lens pin and the ground shield wire on the outside of the coaxial connected to negative polarity lens pin. The lens coaxial cable (not shown in FIG. 21) is routed through main frame wire channel 2105 and lens wire channel 2104. The electrochromic lens 2101 seats inside of main frame lens cavity 2103 which holds the lens in place.

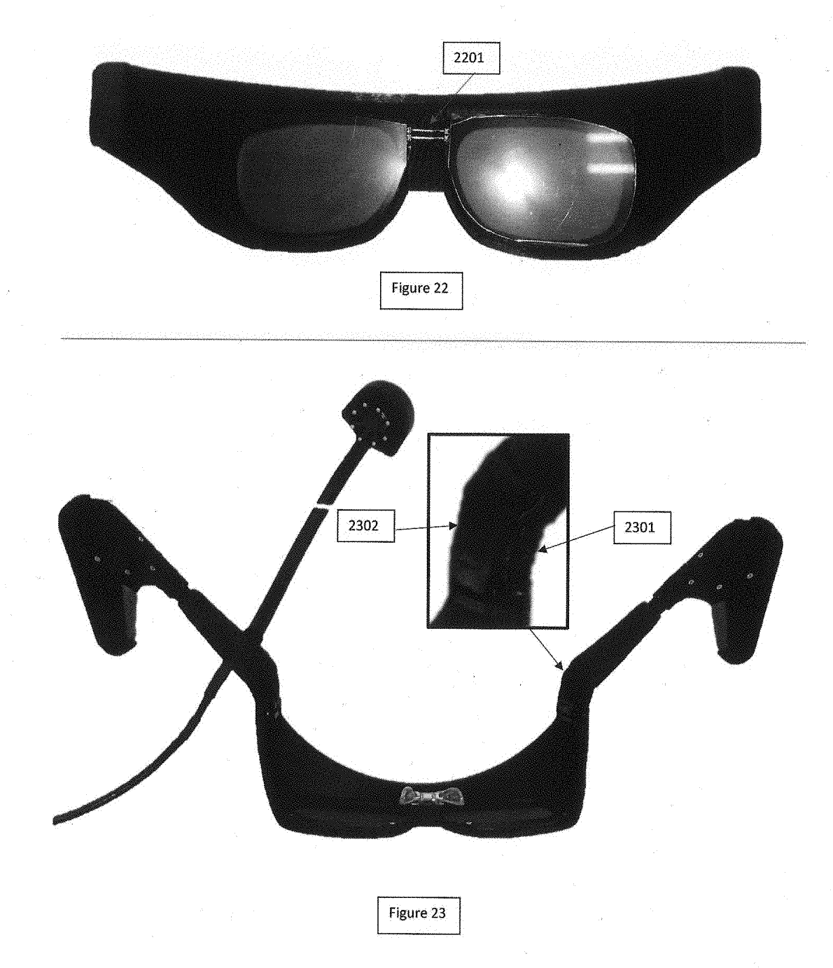

FIG. 22 is an image of the main frame with electrochromic lenses inserted. Lens connector pins 2201 are shown with positive polarity pin of right lens being connected to positive polarity of left lens and negative polarity of right lens being connected to negative polarity of left lens. The lens coaxial cable ground shield is connected to lens negative polarity pin and lens coaxial cable signal wire is attached to positive polarity lens pin. The lens coaxial cable supplies a variable pulse width modulation (PWM) signal at a frequency of 1.2 KHz and a variable voltage of up to 10 V. A higher power signal causes internal liquid crystals inside the glass lens to oscillate, causing the lenses to become more opaque. By removing the power signal, the liquid crystals return to a non-oscillated state, causing lens to return to clear (non-opaque). By varying the intensity of the power signal, various states of opacity are achievable. Those skilled in the art will appreciate that alternative PWM frequencies may be used to electrify the lenses and that the 1.2 KHz signal was adopted to reduce noise infiltration into the EEG signal which occurs at lower frequencies, as well as to inhibit any lens flickering issues which can be seen at lower and higher frequencies.

FIG. 23 is an image of the system with arms opened to full horizontal articulation and a detailed close view of the right hinge in fully opened position. The details close view illustrates the right flexible living hinge in fully opened position. Note the hinge ribs 2301 are fully compressed and hinge inside wall is fully extended. This ability to fully open the hinges and allow vertical and horizontal articulation of the arms is not available with traditional hinge designs.

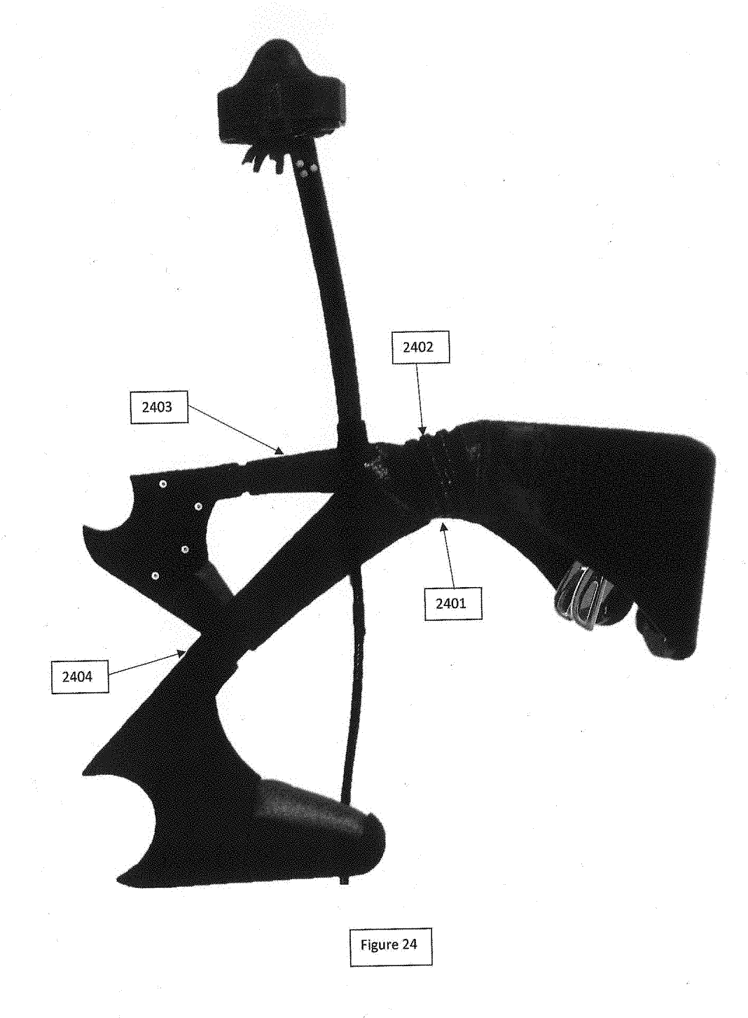

FIG. 24 is an image of the system with arms twisted to full vertical articulation. Left arm 2403 is half-way opened horizontally, as is right arm 2404. Left arm 2403 is positioned horizontal to plane and right arm 2404 is articulated down-ward (vertically) at approximately a 40 degree angle. Note the hinge ribs bottom 2401 are fully compressed and hinge ribs top 2402 are fully extended.

In a different embodiment of the present invention, an alternative micro-spike sensor is presented which also can be used a stimulation probe to induce electrical stimulation through the body.

In one aspect of the present invention a “micro-spike” electrode is designed as a high density of spikes within a small surface area to create a bed-of-nails effect to prevent the spikes from completely breaking through the skin but protrudes enough to reach through the first layers of the epidermis to achieve a low impedance connection to the body.

In one preferred embodiment of the present invention, said micro-spike electrode can be made of any plastic or resin materials or of metallic or other conductive materials. In the case of plastic or 3D printed materials, the sensor can be coated with metallic substrate such as gold or silver to create a low impedance coating for contact with the body. In the case of the exemplary prototype depicted in this description, as well as illustrated in the drawings, a photopolymer 3D printing process was used to form the plastic micro-spike substrate which was then coated with gold plating process. The same or similar sensor design can be directly 3D printed with a metallic, conductive material instead of plating metal on top of non-conductive substrate.

In one aspect of the present invention, said micro-spike sensor can also be used as a stimulation electrode instead of measurement. In the case of stimulation, a signal such as signals seen in TENS units or TDCs is connected to a conductive part of the micro-spike electrode. The signals are transmitted through the conductive surface of the electrode which has a low surface resistivity (<1 ohm/cm2) and through the surface of the micro-spikes which are then transmitted through the epidermis. Let it be known that the exemplary embodiment described herewith illustrates said micro-spike sensor in use on the head. Alternative uses for said sensor are available, such as by example but not limited to, measurement of EMG muscle activity on different parts of the body, or on the chest for EKG measurements, and additionally can be used on various parts of the body as TENS micro current stimulation.

In one preferred embodiment of the present invention said conductive elastomer can be made of silicone or other elastomeric material and contain materials such as but not limited to carbon, silver, gold, aluminum, nickel, graphite nickel plated graphite particles, or any combination thereof to increase the conductivity of the elastomeric substrate.

Those skilled in the art will appreciate that careful measures must be taken in the selection of material composition to reduce galvanization of the material which can occur when combining dissimilar metals and with contact with body sweats and oils which can cause galvanization and changes in PH. Galvanization can cause a high DC voltage offset which can disrupt the measurement of physiologic signals. One measure commonly implemented is the integration of chloride to reduce oxidation and galvanization. This is commonly seen in the case of silver-silver chloride electrodes in which the chloride component is added to reduce galvanization.

In another preferred embodiment of the present invention carbon, aluminum, silver, gold in any combination can be used to achieve high surface conductivity. It should be noted that some materials have higher biocompatibility than other materials such as nickel or aluminum which certain people can have negative reactions to such as rashes or blisters. In this case, it is important to manage the ratio of the reactive materials within the compound and to take preventative measures in design of the sensors and manufacturing to limit the amount of the reactive materials on the surface and within the composite materials.

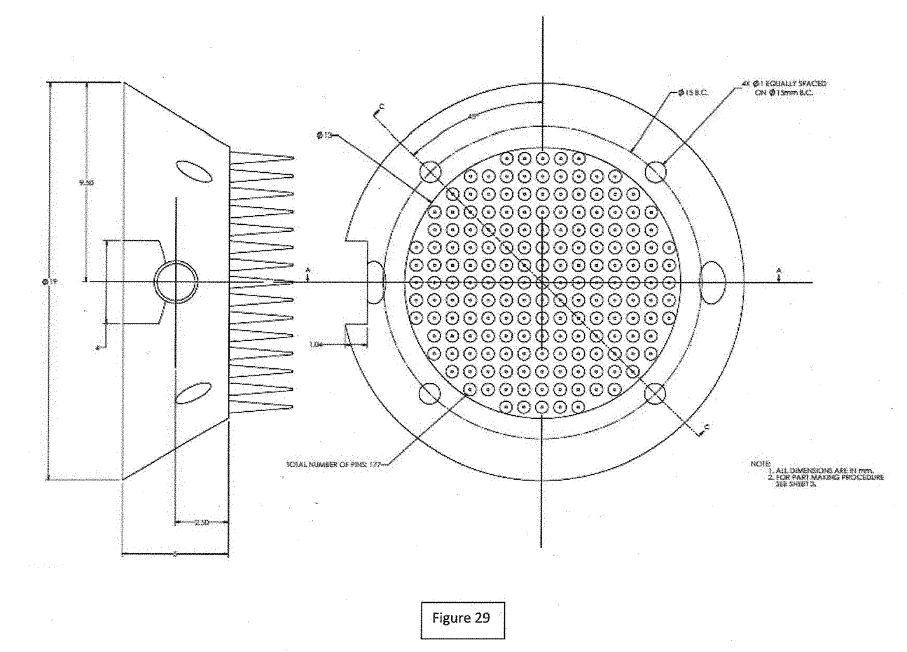

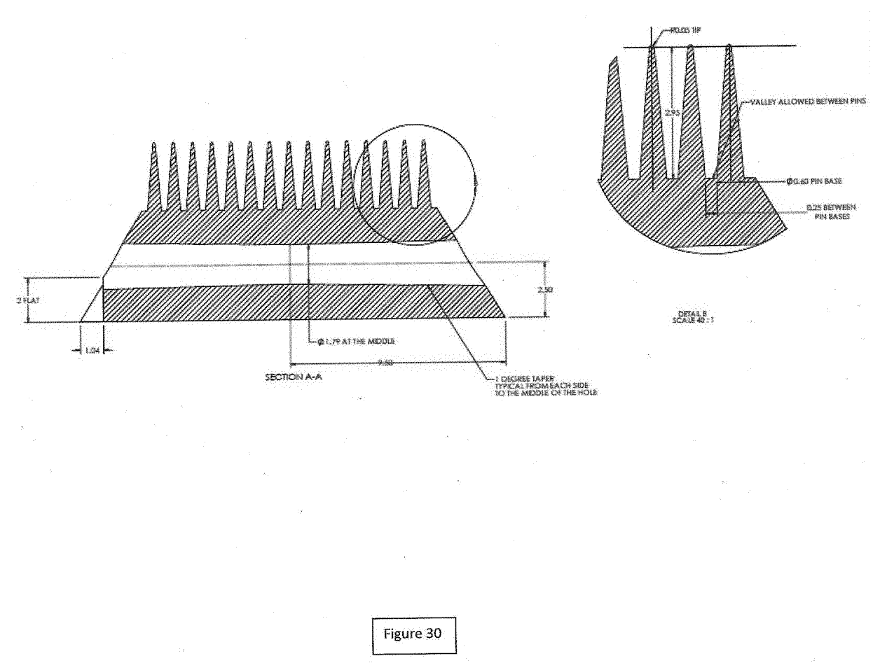

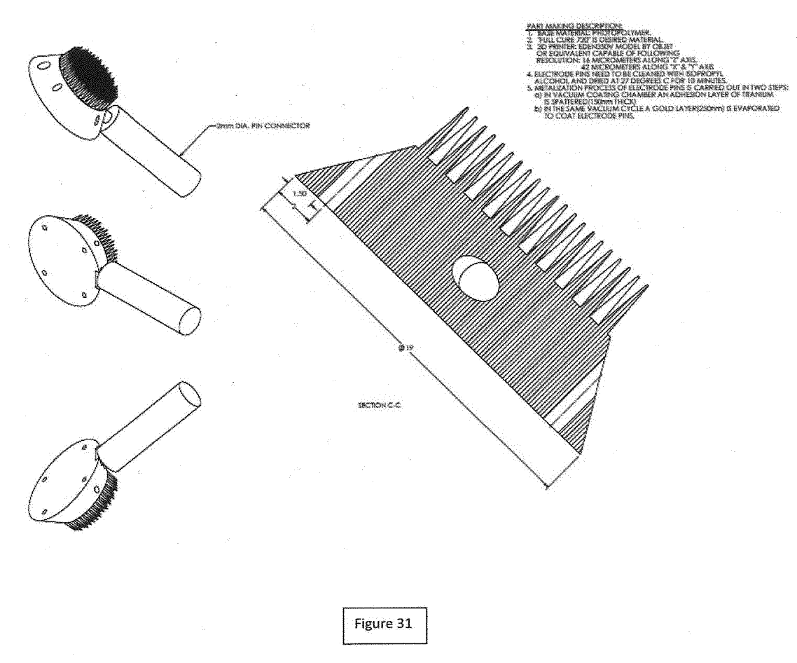

Referring now to FIG. 29, FIG. 30, and FIG. 31, engineering drawings illustrate the dimensions of said micro-spike sensing/stimulating probe.

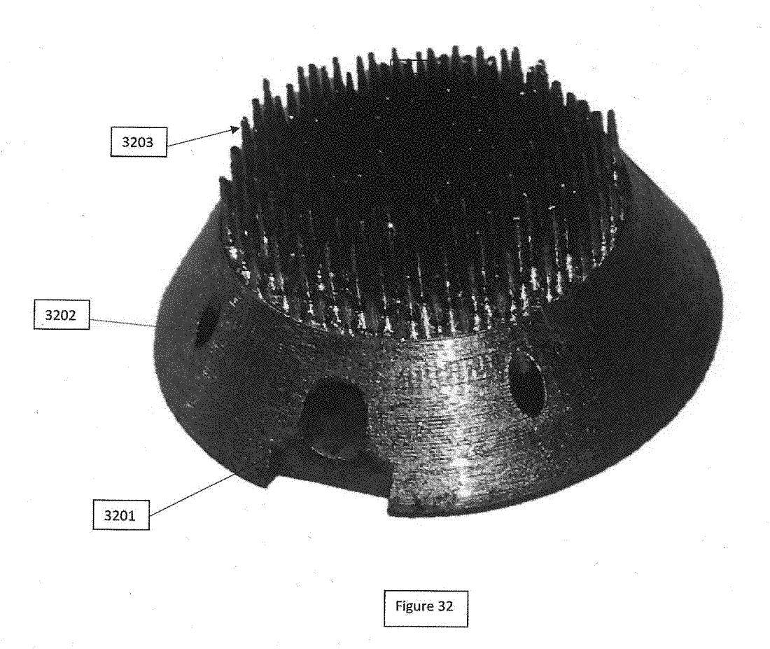

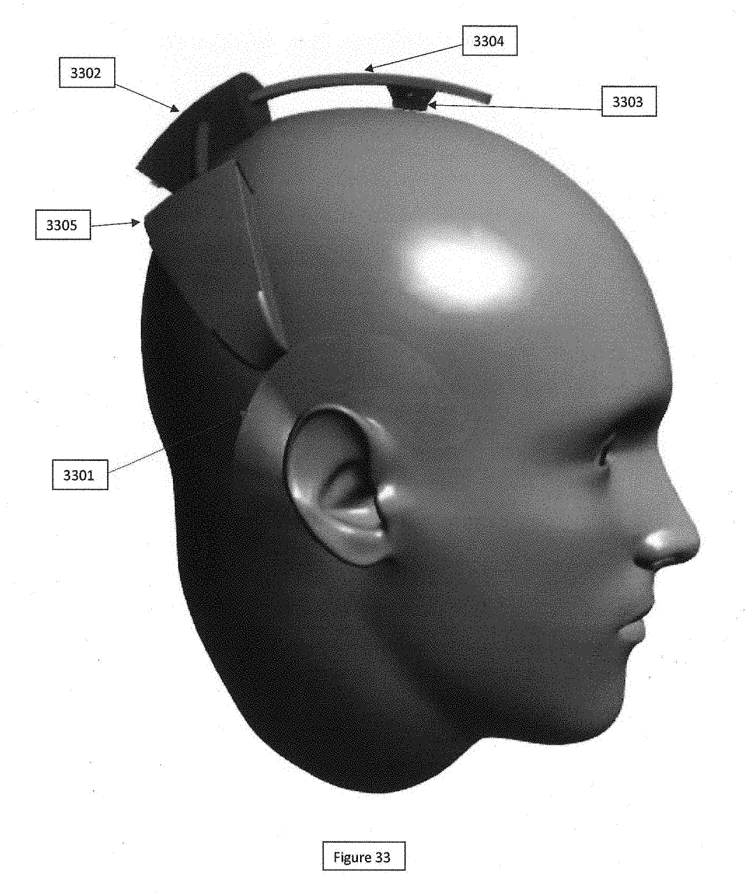

Referring now to FIG. 32, an image of said micro-spike sensing/stimulating probe is presented which is a physical prototype of said micro-spike as illustrated in FIG. 29, FIG. 30, and FIG. 31. Connector hole 3201 allows for a connector to be inserted to create electrical continuity to the apparatus. Screw hole 3202 allows for screws to be inserted to mount said micro-spike on an additional apparatus. There are 144 micro-spikes 3203 in said device. Those skilled in the art will appreciate that this prototype was manufactured using 3D printing with photopolymers, then coated with gold plating technique to create an ultra-low surface resistivity.

In a different embodiment of the present invention, an alternative headset design is presented which can be used with said micro-spike device. Ear piece 3301 allows for placement on the head, around the top of the ears. Main body 3302 allows for top arm 3304 to slide forward and backward to allow for different placement of micro-spike 3303. Side piece 3305 is allowed to slide laterally to adjust to different head sizes and shapes.

It will be appreciated that numerous other such modifications and variations of the illustrated embodiments are possible, and it is therefore intended that the invention be limited solely in accordance with the appended claims.

What is claimed is:

1. Apparatus for obtaining EEG signals by way of adjustable arms and EEG sensors, wherein the adjustable arms and EEG sensors are mounted on an adjustable frame adapted to fit the head of an individual.
